(19) United States
(12) Patent Application Publication
(10) Pub. No.: US 2017/0281418 A1
(43) Pub. Date: Oct. 5, 2017

(54) SHEET-LIKE WATER-ABSORBING STRUCTURE

(71) Applicant: GARRY TSAUR, ROWLAND HEIGHTS, CA (US)

(72) Inventor: GARRY TSAUR, ROWLAND HEIGHTS, CA (US)

(21) Appl. No.: 15/474,123

(22) Filed: Mar. 30, 2017

(30) Foreign Application Priority Data
Mar. 30, 2016 (TW) 105204406

(51) Int. Cl. A61F 13/475; A61L 15/18; A61F 13/84; A61F 13/62; A61F 13/64; A61L 15/38; B01J 20/04

(52) U.S. Cl. CPC A61F 13/4758; A61L 15/38; A61L 15/18; C12Y 111/01006; B01J 20/041; A61F 13/622; A61F 13/64; A61F 13/8405

(57) ABSTRACT

A sheet-like water-absorbing structure is provided. The aforementioned structure includes: a sheet-like body that includes an absorptive object and elastic material. Meanwhile, the elastic material surrounds the sheet-like body. The fore-and-aft length of the aforementioned structure is longer than its left-and-right width. Two symmetric first connected structures are located at the front end of the aforementioned structure and two symmetric second one are located at the rear end of the aforementioned structure. Meanwhile, the aforementioned structure has no adhesive materials and it relying on the human body by the first connected structure and the second connected structure.

### BACKGROUND OF THE INVENTION

1. Field of the invention

The present invention generally relates to a sheet-like water-absorbing structure, e.g. sanitary napkin, and it can be fastened by the elastic strip structures at its four ends, but not by the adhesive to stick to the underpants.

2. Description of the prior art

The Sanitary napkin is a special product for female menstrual cycle, which can keep body clean. Most sanitary napkins in the past stuck to the underpants with the adhesive. In 1921, Kimberly-Clark Company of America created the first disposable sanitary napkin, which should be fastened to the underpants or clothes with pins. Until 1970s, the self-adhesion gum design was born. However, the design to sanitary napkin until this day doesn't break the conception of fastening to the underpants with self-adhesion gum.

The binding degree between sanitary napkin and the body determines whether the menstrual blood will be leaking, which directly affects the comfort degree and cleanliness. Even though the sanitary napkin that fastened to the underpants with self-adhesion gum, can be perfectly stuck to the underpants, its binding degree with the body still be affected by the elasticity, tightness & cutting method and others of underpants.

Some women like to choose dipper (pants) in their physiology period in order to avoid the leakage of menstrual blood. However, as the dipper (pants) can totally cover the waist &abdomen, private parts and buttocks, it will make them look like so fat. In addition, its predicted liquid absorption volume is bigger than that of general sanitary napkins, which makes a bad comfort degree and cause the users to feel airtight and stuffy. Moreover, the dipper (pants) with elastic belt around the waist can be only removed by taking off the outer pants first, which has no conveniences. If sticking the two sides of dipper (pants) with Velcro, then it may even break women's delicate clothes.

As it covers so much of the body, sanitary napkin whether adhering to underpants or in dipper (pants)-like design has a series of disadvantages, such as making users feel stuffy and not dry, creating larger impression, without any beauty sense by the outer clothes covered places bulging, damaging cloths at adhesion place, etc. Therefore, it becomes an important task for the present invention to design a sheet-like water-absorbing structure that not adhering to the underpants, not making users feel stuffy and fat and not damaging cloths.

### SUMMARY OF THE INVENTION

The purpose of the present invention is to provide a sheet-like water-absorbing structure, which contains: a sheet-like body that includes an absorptive object and elastic material. Meanwhile, the elastic material surrounds the sheet-like body. The fore-and-aft length of this sheet-like water absorbing structure is longer than its left-and-right width. Two symmetric first connected structures are located at the front or back end of the sheet-like body and two symmetric second connected structures are located at the front or back end of the sheet-like body. Meanwhile, the sheet-like water-absorbing structure has no adhesive materials and it relying on human body by the first connected structure and the second connected structure.

In order to achieve the aim of the above-mentioned invention, the first connected structure is in circle or hole shaped and the second connected structure is in elastic long-strip form.

In order to achieve the aim of the above-mentioned invention, the elastic long-strip structure is equipped with hooks.

In order to achieve the aim of the above-mentioned invention, the elastic long-strip structure is also equipped with snap fasteners, which contains one or several male snaps and one or several female snaps.

In addition, in order to achieve the aim of the above-mentioned invention, the elastic long-strip structure is equipped with button or fastener.

In order to achieve the aim of the above-mentioned invention, the first connected structures is an elastic long-strip structure, the second connected structures is an elastic long-strip structure.

In order to achieve the aim of the above-mentioned invention, the sheet-like body can further contain oxygen- release material, wherein said oxygen- release material belongs to peroxide metal or superoxide metal; wherein the peroxide metal is selected from the group consisting magnesium peroxide, calcium peroxide, sodium peroxide, potassium peroxide and lithium peroxide; wherein the superoxide metal is selected from the group consisting magnesium superoxide, calcium superoxide, sodium superoxide, potassium superoxide and lithium superoxide.

Meanwhile, in order to achieve the aim of the above-mentioned invention, the sheet-like body can further contain negative oxygen ion-release material, wherein said negative oxygen ion-release material belongs to tourmaline combined with peroxide metal or superoxide metal; wherein the peroxide metal is selected from the group consisting magnesium peroxide, calcium peroxide, sodium peroxide, potassium peroxide and lithium peroxide; while the superoxide metal is selected from the group consisting magnesium superoxide, calcium superoxide, sodium superoxide, potassium superoxide and lithium superoxide.

Meanwhile, in order to achieve the aim of the above-mentioned invention, the sheet-like body can further contain oxygen-rapid release material, which belongs to using catalase in combination with peroxide metal or superoxide metal; wherein the peroxide metal is selected from the group consisting magnesium peroxide, calcium peroxide, sodium peroxide, potassium peroxide and lithium peroxide; wherein the superoxide metal is selected from the group consisting magnesium superoxide, calcium superoxide, sodium superoxide, potassium superoxide and lithium superoxide.

Besides, in order to achieve the aim of the above-mentioned invention, the ratio of catalase to peroxide metal or superoxide metal is 1:50˜1:1000.

Moreover, in order to achieve the aim of the above-mentioned invention, the sheet-like water-absorbing structure can be produced as panty liners, sanitary napkins or puerperal liners.

Finally, in order to achieve the aim of the above-mentioned invention, the sheet-like water-absorbing structure shall contain at least one liquid-tight sheet.

### BRIEF DESCRIPTION OF THE DRAWINGS

FIG. 1 is a schematic view of a sanitary napkin with circle-shaped structure and elastic strip-like structure with snap fasteners.

FIG. 2 is a usage schematic view of a sanitary napkin with circle-shaped structures and elastic strip-like structures with snap fasteners and then the male snaps fastening to the female snaps after the latter structure crossing the former one.

FIG. 3 is a schematic view of a puerperal liner with elastic strip-like structures

FIG. 4 is a usage schematic view of a panty liner with holes and elastic strip-like structures and then the latter structure crossing the former one for fixation.

FIG. 5 is a usage schematic view of a sanitary napkin with circle-shaped structures and elastic strip-like structures and then the latter structure crossing the former one for fixation.

FIG. 6 is a usage schematic view of a sanitary napkin with circle-shaped structures and elastic strip-like structures with buttons or fasteners and then buttons or fasteners fastening with each other after the latter structure crossing the former one.

FIG. 7 is a usage schematic view of a sanitary napkin with circle-shaped structures and elastic strip-like structures with hooks and then the hooks clasping the circle-shaped structures for fixation.

FIG. 8 is a usage schematic view of a sanitary napkin and then the male snaps fastening to the female snaps after the elastic strip-like materials easily crossing the snap rings for fixation at users' thighs as they are sitting or in half a squat.

### DESCRIPTION OF MAJOR COMPONENT SYMBOLS

| | |
|---|---|
| 1 | Sheet-like Body |
| 101 | Front end of sheet-like body |
| 102 | Rear-end of sheet-like body |
| 2 | Water-absorbing material |
| 3 | Elastic material |
| 4 | Snap Ring |
| 5 | Elastic strip-like material |
| 6 | Male Snap |
| 7 | Female Snap |
| 8 | Hole |
| 9 | Fastener |
| 10 | Hook |

### DETAILED DESCRIPTION OF THE PREFERRED EMBODIMENT

The present invention will be demonstrated through the following embodiments but not be subject to them.

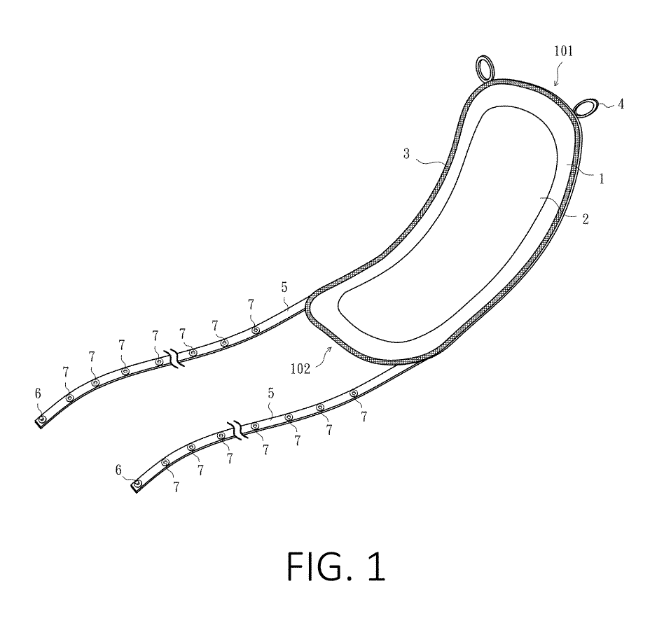

This present invention is a kind of sanitary napkin without sticking to underpants. Refer to FIG. 1, the sheet-like body (1) contains one water-absorbing material (2), which is surrounded with elastic material (3). Connect the snap ring (4) of the first connected structure at the front end (101)of the sanitary napkin to the elastic strip-like material (5) with male snaps (6) and female snaps (7) of the second connected structure at the rear-end (102) of the sanitary napkin and then fasten the male snaps with female snaps so as to make the elastic strip-like material be tightly attached to users' waist-hip position and then the sanitary napkin will be fully fit for their bodies.

### Embodiment 1

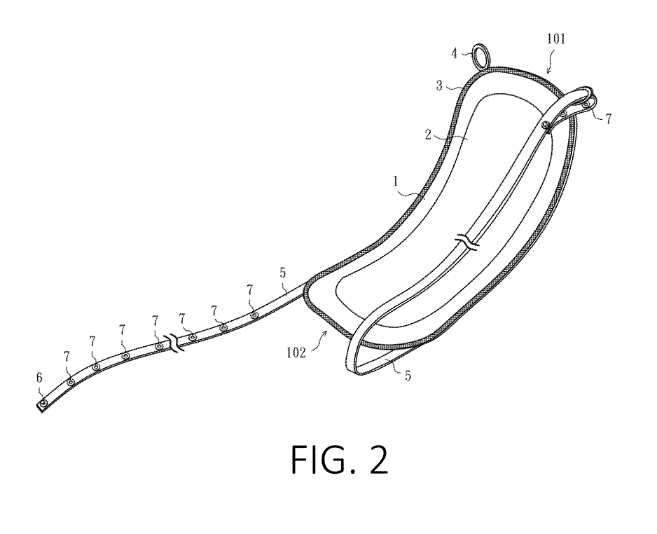

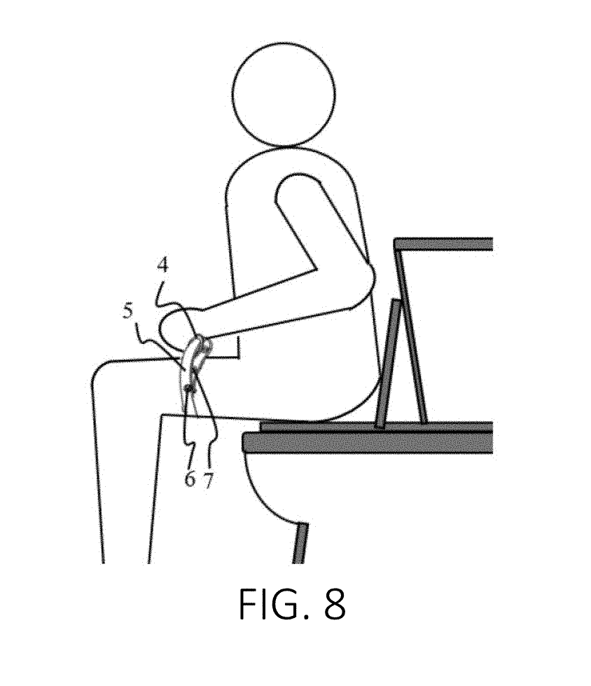

The sheet-like water-absorbing structure, such as the sanitary napkin as shown in (FIG. 1) contains one sheet-like body(1) and the sheet-like body further contains one water-absorbing material(2), which is surrounded with elastic material(3). There are two snap rings (4)of the first connected structure at both ends of the front end(101)of the sheet-like body and there are two responding elastic strip-like materials with male snaps(6) and female snaps (7) of the second connected structure at both ends of the rear-end (102)of the sheet-like body. Meanwhile, there is at least one male snap and female snap. Cross the elastic strip-like materials through the snap rings as wearing it as shown in FIG. 2 and fasten the male snap with the female snap based on users' waist-hip circumference(FIG. 2) so as to make the elastic strip-like material be tightly attached to users' waist-hip position and then the sanitary napkin will be fully fit for their bodies. Therefore, the sanitary napkin will be in suitable tightness, and breathable and comfortable without any leakage of secreta and menstrual blood. The fore-and-aft length of the sanitary napkin is about 15-45 centimeters and its width is about 5-20 centimeters. Users can make sitting, squat, half a squat or standing and other postures while wearing the sanitary napkin. Moreover, Especially as sitting or in half a squat, users can easily cross the elastic strip-like materials through snap rings and then fasten the male snaps with female snaps at their thighs without putting on or off their clothes(as shown in FIG. 8).

### Embodiment 2

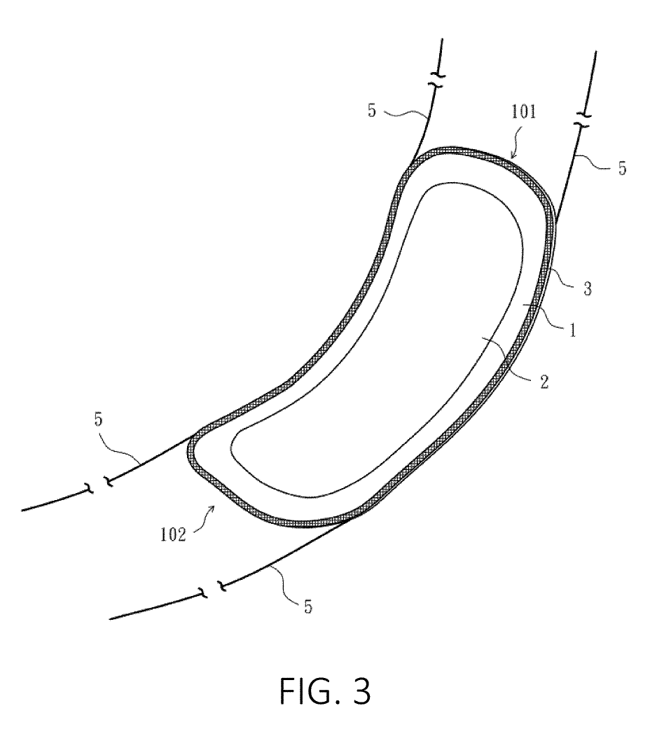

The sheet-like water-absorbing structure, such as the puerperal liner as shown in (FIG. 3) contains one sheet-like body(1) and the sheet-like body further contains one water-absorbing material(2), which is surrounded with elastic material(3). There are the first and second connected structures respectively located at both ends of the front end (101)of and the rear-end of the sheet-like body and both of these two above-mentioned structures belong to elastic materials (5). Tie the elastic strip-like material at the front end of sheet-like body to the corresponding one that at the rear-end of sheet-like body as wearing so as to make the elastic strip-like material be tightly attached to users' waist-hip position and then the puerperal liner will be fully fit for their bodies. Therefore, the puerperal liner will be in suitable tightness, and breathable and comfortable without any leakage of secreta and menstrual blood. The optimum length of the puerperal liner is about 30-60 centimeters and its optimum width is about 5-40 centimeters. Users can make sitting, squat, half a squat or standing and other postures while wearing the puerperal liner. Moreover, Especially as sitting or in half a squat, users can easily knot the elastic strip-like materials at their thighs without putting on or off their clothes.

### Embodiment 3

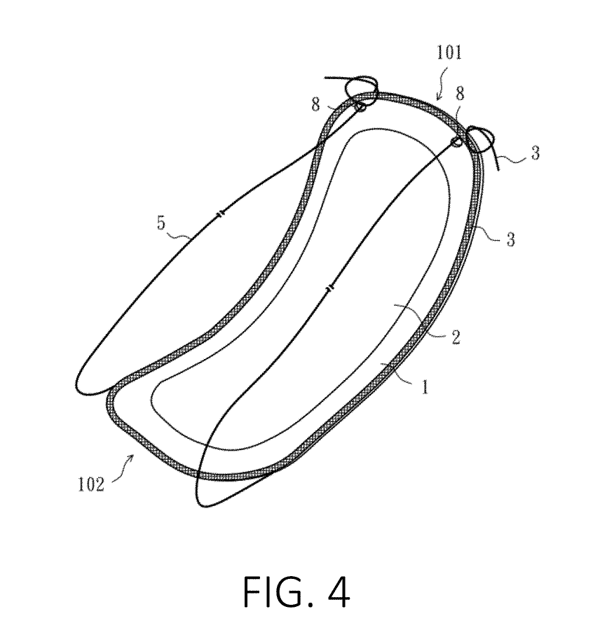

The sheet-like water-absorbing structure, such as the panty liner as shown in (FIG. 4) contains one sheet-like body(1) and the sheet-like body further contains one water-absorbing material(2), which is surrounded with elastic material(3).

There are two holes (8)of the first connected structure at both ends of the front end(101)of the sheet-like body and there are two responding elastic strip-like materials of the second connected structure at both ends of the rear-end (102)of the sheet-like body. Cross the elastic strip-like materials through the snap rings as wearing it based on users' waist-hip circumference so as to make the elastic strip-like material be tightly attached to users' waist-hip position and then the panty liner will be fully fit for their bodies. Therefore, the panty liner will be in suitable tightness, and breathable and comfortable without any leakage of secreta and menstrual blood. The optimum fore-and-aft length of the panty liner is about 8-20 centimeters and its optimum width is about 1-10 centimeters. Users can make sitting, squat, half a squat or standing and other postures while wearing the panty liner. Moreover, Especially as sitting or in half a squat, users can easily cross the elastic strip-like materials through holes for knots at their thighs without putting on or off their clothes.

### Embodiment 4

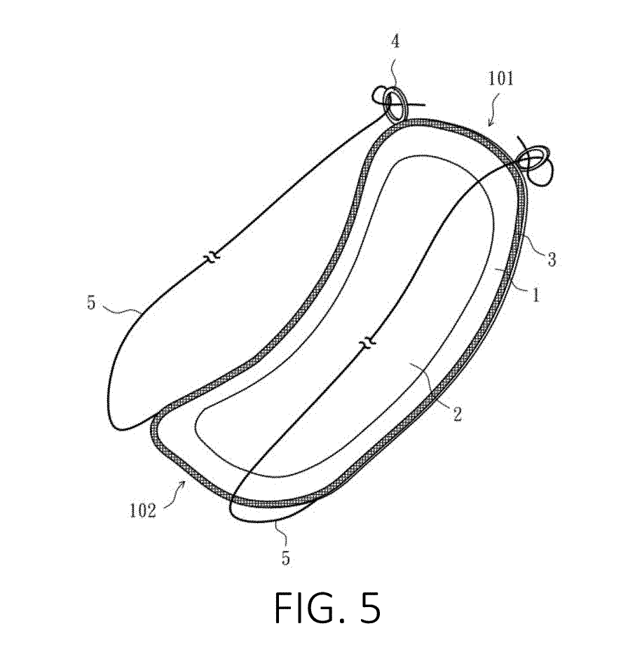

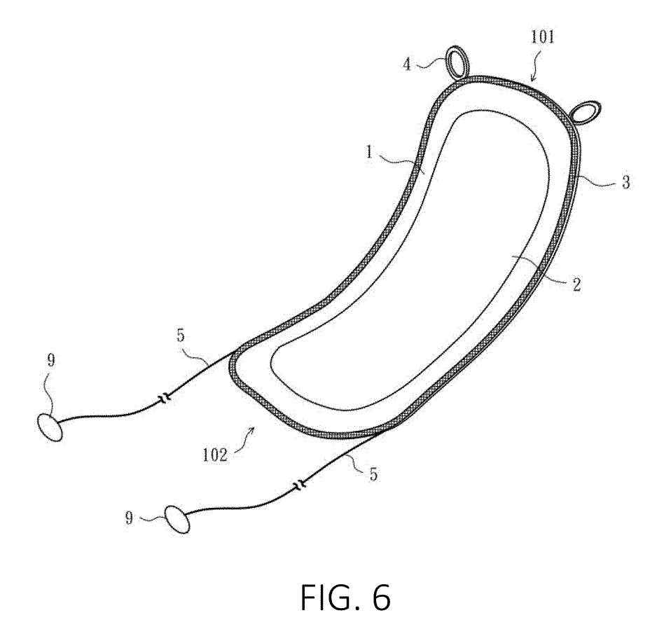

The sheet-like water-absorbing structure, such as the sanitary napkin as shown in (FIG. 5) contains one sheet-like body(1) and the sheet-like body further contains one water-absorbing material(2), which is surrounded with elastic material(3). There are two hole rings (4)of the first connected structure at both ends of the front end(101)of the sheet-like body and there are two responding elastic strip-like materials (5) of the second connected structure at both ends of the rear-end (102)of the sheet-like body. Cross the elastic strip-like materials through the hole rings for knots as wearing it or cross the fasteners on the elastic strip-like materials (if any) (FIG. 6) through the hole rings based on users' waist- hip circumference so as to make the elastic strip-like material be tightly attached to users' waist-hip position and then the sanitary napkin will be fully fit for their bodies. Therefore, the sanitary napkin will be in suitable tightness, and breathable and comfortable without any leakage of secreta and menstrual blood. Users can make sitting, squat, half a squat or standing and other postures while wearing the sanitary napkin. Moreover, Especially as sitting or in half a squat, users can easily cross the fasteners on elastic strip-like materials through hole rings for fixation at their thighs without putting on or off their clothes.

### Embodiment 5

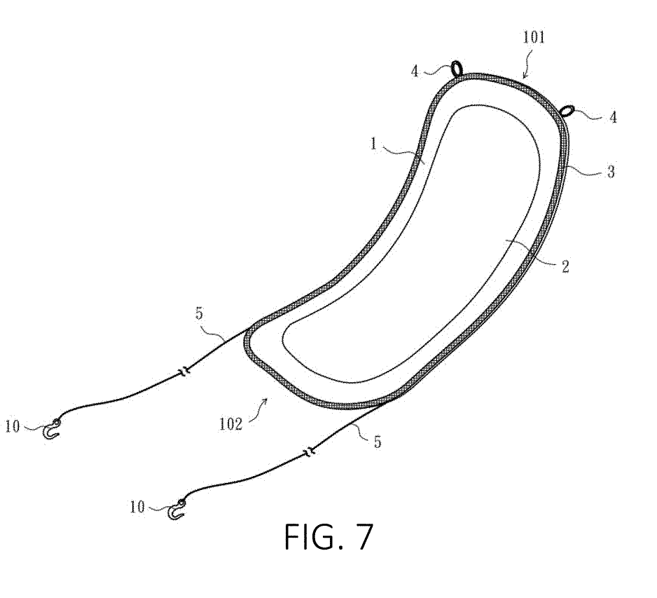

The sheet-like water-absorbing structure, such as the sanitary napkin as shown in (FIG. 7) contains one sheet-like body(1) and the sheet-like body further contains one water-absorbing material(2) , which is surrounded with elastic material(3). There are two hole rings (4)of the first connected structure at both ends of the front end(101)of the sheet-like body and there are two responding elastic strip-like materials (5) with hooks (10)of the second connected structure at both ends of the rear-end (102)of the sheet-like body. Clasp the hooks (10) at the elastic strip-like materials with the hole rings(4) as wearing it based on users' waist- hip circumference so as to make the elastic strip-like material be tightly attached to users' waist-hip position and then the sanitary napkin will be fully fit for their bodies. Therefore, the sanitary napkin will be in suitable tightness, and breathable and comfortable without any leakage of secreta and menstrual blood. Users can make sitting, squat, half a squat or standing and other postures while wearing the sanitary napkin. Moreover, Especially as sitting or in half a squat, users can easily clasp the hooks on elastic strip-like materials with the hole rings at their thighs without putting on or off their clothes.

### Embodiment 6

Sheet-like water-absorbing structure contains oxygen-release material.

2KO₂+2H₂O→2KOH+H₂O₂+O₂

The sheet-like water-absorbing structure, such as sanitary napkin, also contains potassium superoxide particles in addition to absorptive components. As potassium superoxide may produce potassium hydroxide and oxygen after reacting with the moisture in the menstrual blood. Moreover, it can also produce oxygen after the potassium superoxide mentioned above reacts with the moisture in the air, which can largely increase the oxygen concentration in the sanitary napkin.

### Embodiment 7

Sheet-like water-absorbing structure contains negative oxygen ion-release material.

2MgO₂+2H₂O→2Mg(OH)₂+O₂e⁻+O₂→O₂⁻

It can produce negative oxygen ion through using magnesium peroxide in combination with tourmaline to react with the moisture in the menstrual blood. The sheet-like water-absorbing structure, such as sanitary napkin, also contains magnesium peroxide powder and tourmaline powder in addition to absorptive components. Peroxide such as magnesium peroxide may produce potassium hydroxide and oxygen after reacting with the moisture in the menstrual blood. Moreover, it can also produce oxygen after the magnesium peroxide mentioned above reacts with the moisture in the air. Meanwhile, it can create negative oxygen ion as negative charge produced by tourmaline reacts with the oxygen in the air, which can largely increase the negative oxygen ion concentration in the sanitary napkin.

### Embodiment 8

Sheet-like water-absorbing structure contains oxygen-rapid release material.

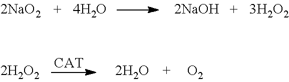

It can rapidly produce oxygen through using peroxide metal, such as potassium peroxide, in combination with catalase (CAT for short) to react with water. In addition to absorptive components, the sheet-like water-absorbing structure, such as sanitary napkin, also contains potassium peroxide powder and catalase powder in ratio of 50:1˜1000:1. Hereinto, their best radio is 100:1˜500:1. Superoxide such as sodium superoxide (NaO₂) may produce hydrogen peroxide after reacting with the moisture in the menstrual blood. After that, it can rapidly produce oxygen after catalase (CAT for short) reacts with the above-created hydrogen peroxide in a short time, which can largely increase the oxygen concentration in the sanitary napkin.

While the means of specific embodiments in present invention has been described by reference drawings, numerous modifications and variations could be made thereto by those skilled in the art without departing from the scope and spirit of the invention set forth in the claims. The modifications and variations should in a range limited by the specification of the present invention.

In view the above-mentioned functions fully conform to inventiveness and advancement essentially and necessarily required by the legal patent, now apply to your bureau for approval in accordance with relevant laws and we cordially invite you to approve this patent application to encourage our invention.

What is claimed is:

1. A sheet-like water-absorbing structure comprising:
a sheet-like body includes an absorptive object and elastic material, wherein the elastic material surrounds the sheet-like body, the fore-and-aft length of
said sheet-like water absorbing structure is longer than its left-and-right width;
two symmetric first connected structure are located at the front or back end of the sheet-like body;
two symmetric second connected structure are located at the sheet-like body in the same way;
wherein said sheet-like water-absorbing structure has no adhesive materials, wherein said sheet-like water-absorbing structure relying on human body by the first connected structure and the second connected structure.

2. The sheet-like water-absorbing structure in accordance with claim 1, wherein the first connected structure is in circle or hole shaped and the second connected structure is in elastic long-strip form.

3. The sheet-like water-absorbing structure in accordance with claim 2, wherein the elastic long-strip structure is equipped with hooks.

4. The sheet-like water-absorbing structure in accordance with claim 2, wherein the elastic long-strip structure is also equipped with snap fasteners, which contains one or several male snaps and one or several female snaps.

5. The sheet-like water-absorbing structure in accordance with claim 2, wherein the elastic long-strip structure is equipped with button or fastener.

6. The sheet-like water-absorbing structure in accordance with claim 1, wherein the first connected structure is in elastic long-strip structure, wherein the second connected structure is in elastic long-strip structure.

7. The sheet-like water-absorbing structure in accordance with claim 1, wherein the sheet-like body can further contain oxygen- release material, which belongs to peroxide metal or superoxide metal.

8. The sheet-like water-absorbing structure in accordance with claim 7, wherein the peroxide metal is selected from the group consisting magnesium peroxide, calcium peroxide, sodium peroxide, potassium peroxide and lithium peroxide.

9. The sheet-like water-absorbing structure in accordance with claim 7, wherein the superoxide metal is selected from the group consisting magnesium superoxide, calcium superoxide, sodium superoxide, potassium superoxide and lithium superoxide.

10. The sheet-like water-absorbing structure in accordance with claim 1, wherein the sheet-like body can further contain negative oxygen ion-release material, wherein said negative oxygen ion-release material is tourmaline combined with peroxide metal or superoxide metal.

11. The sheet-like water-absorbing structure in accordance with claim 10, wherein the peroxide metal is selected from the group consisting magnesium peroxide, calcium peroxide, sodium peroxide, potassium peroxide and lithium peroxide

12. The sheet-like water-absorbing structure in accordance with claim 10, wherein the superoxide metal is selected from the group consisting magnesium superoxide, calcium superoxide, sodium superoxide, potassium superoxide and lithium superoxide.

13. The sheet-like water-absorbing structure in accordance with claim 1, wherein the sheet-like body can further contain oxygen-rapid release material, which belongs to using catalase in combination with peroxide metal or superoxide metal.

14. The sheet-like water-absorbing structure in accordance with claim 13, wherein the ratio of catalase to peroxide metal or superoxide metal is 1:50˜1:1000.

15. The sheet-like water-absorbing structure in accordance with claim 13, wherein the peroxide metal is selected from the group consisting magnesium peroxide, calcium peroxide, sodium peroxide, potassium peroxide and lithium peroxide

16. The sheet-like water-absorbing structure in accordance with claim 13, wherein the superoxide metal is selected from the group consisting magnesium superoxide, calcium superoxide, sodium superoxide, potassium superoxide and lithium superoxide.

17. The sheet-like water-absorbing structure in accordance with claim 1, wherein said sheet-like water-absorbing structure is panty liner, sanitary napkin or puerperal liner.

18. The sheet-like water-absorbing structure in accordance with claim 1, wherein said sheet-like water-absorbing structure contains at least one liquid-tight sheet.
